SEQUENCE LISTING (10 sequences)

SEQ ID NO: 1 - DNA, 294 bp, synthetic construct
  source 1..294: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaa

SEQ ID NO: 2 - DNA, 393 bp, synthetic construct
  source 1..393: mol_type = other DNA
atgcacaaatcttctccgcagggtccggatcgtctgctgatccgtctgcgtcacctgatcgatatcgttgaacagctgaaaatctacgaaaacgacctggatccggaactgctgtctgctccgcaggatgttaaaggtcactgcgaacacgctgctttcgcgtgtttccagaaagctaaactgaaaccgtctaatccgggtaacaacaaaaccttcatcatcgacctggttgcgcagctgcgtcgtcgtctgccggctcgtcgtggtggtaaaaaacagaaacacatcgctaaatgcccgtcttgtgactcttacgaaaaacgtaccccgaaagaattcctggaacgtctgaaatggctgctgcagaaaatgatccaccagcacctgtcttaa

SEQ ID NO: 3 - DNA, 957 bp, synthetic construct
  source 1..957: mol_type = other DNA
aggaggtttgatcctatgggaacaatgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 4 - DNA, 48 bp, synthetic construct
  source 1..48: mol_type = other DNA
ggcggcagcagccatcaccatcaccatcaccatcacagcagcggcggc

SEQ ID NO: 5 - DNA, 42 bp, synthetic construct
  source 1..42: mol_type = other DNA
ggcggcagcagccatcaccatcaccatcacagcagcggcggc

SEQ ID NO: 6 - DNA, 735 bp, synthetic construct
  source 1..735: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcaccatcacagcagcggcggcatgcacaaatcttctccgcagggtccggatcgtctgctgatccgtctgcgtcacctgatcgatatcgttgaacagctgaaaatctacgaaaacgacctggatccggaactgctgtctgctccgcaggatgttaaaggtcactgcgaacacgctgctttcgcgtgtttccagaaagctaaactgaaaccgtctaatccgggtaacaacaaaaccttcatcatcgacctggttgcgcagctgcgtcgtcgtctgccggctcgtcgtggtggtaaaaaacagaaacacatcgctaaatgcccgtcttgtgactcttacgaaaaacgtaccccgaaagaattcctggaacgtctgaaatggctgctgcagaaaatgatccaccagcacctgtcttaa

SEQ ID NO: 7 - DNA, 729 bp, synthetic construct
  source 1..729: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcacagcagcggcggcatgcacaaatcttctccgcagggtccggatcgtctgctgatccgtctgcgtcacctgatcgatatcgttgaacagctgaaaatctacgaaaacgacctggatccggaactgctgtctgctccgcaggatgttaaaggtcactgcgaacacgctgctttcgcgtgtttccagaaagctaaactgaaaccgtctaatccgggtaacaacaaaaccttcatcatcgacctggttgcgcagctgcgtcgtcgtctgccggctcgtcgtggtggtaaaaaacagaaacacatcgctaaatgcccgtcttgtgactcttacgaaaaacgtaccccgaaagaattcctggaacgtctgaaatggctgctgcagaaaatgatccaccagcacctgtcttaa

SEQ ID NO: 8 - DNA, 58 bp, synthetic construct
  source 1..58: mol_type = other DNA
gatcgatcgagctcaggaggtttgatcctatgggaacaatgcatacatccgagttgct

SEQ ID NO: 9 - DNA, 49 bp, synthetic construct
  source 1..49: mol_type = other DNA
gatcgatcgtcgacttaaacagcctgttcgatctgttcatccagcagtt

SEQ ID NO: 10 - DNA, 1107 bp, synthetic construct
  source 1..1107: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtag

(19) United States
(12) Patent Application Publication
(10) Pub. No.: US 2026/0041735 A1
(43) Pub. Date: Feb. 12, 2026

(54) RECOMBINANT EXPRESSION VECTOR FOR SECRETION OF INTERLEUKIN-21, AND ATTENUATED SALMONELLA STRAIN TRANSFORMED BY MEANS OF SAME

(71) Applicant: THE INDUSTRY & ACADEMIC COOPERATION IN CHUNGNAM NATIONAL UNIVERSITY (IAC), Daejeon (KR)

(72) Inventors: Hyo Jin LEE, Daejeon (KR); Heung Jin JEON, Daejeon (KR); Solbi KIM, Daejeon (KR); Eunji KIM, Daejeon (KR); Minju HAN, Chungcheongbuk-do (KR)

(73) Assignee: THE INDUSTRY & ACADEMIC COOPERATION IN CHUNGNAM NATIONAL UNIVERSITY (IAC), Daejeon (KR)

(21) Appl. No.: 18/998,421

(22) PCT Filed: Jul. 25, 2023
(86) PCT No.: PCT/KR2023/010735

§ 371 Date: Jan. 24, 2025

(30) Foreign Application Priority Data
Jul. 25, 2022 (KR) 10-2022-0092017

(51) Int. Cl. A61K 38/20; A61K 35/74; A61K 48/00; A61P 35/00; C12N 15/74

(52) U.S. Cl. CPC A61K 38/20; A61K 35/74; A61K 48/0058; A61P 35/00; C12N 15/74; C12N 2800/101; C12N 2830/002

(57) ABSTRACT

The present disclosure relates to a recombinant expression vector for secretion of interleukin-21 and an attenuated Salmonella strain transformed therewith, and provides: a recombinant expression vector comprising an flgM gene and an interleukin-21 gene; an attenuated Salmonella strain transformed therewith; and a pharmaceutical composition for cancer treatment, comprising the attenuated Salmonella strain.

### TECHNICAL FIELD

The present disclosure relates to a recombinant expression vector for secretion of Interleukin-21 and an attenuated Salmonella strain transformed therewith. The present application claims priority to Korean Patent Application No. 10-2022-0092017, filed on Jul. 25, 2022, in the Korean Intellectual Property Office, the disclosure of which is incorporated herein by reference.

### BACKGROUND ART

Cancer is a disease that ultimately threatens life as cells continue to proliferate, invade surrounding tissues, and destroy normal cells. As the body's regulatory ability declines with age, it becomes more vulnerable to cancer, and in an aging society, cancer remains the leading cause of death, more than double the second leading cause, heart disease.

The most effective response system for treating disease is to strengthen immunity, but cancer cells are not foreign invaders, so they evade the body's immune response without triggering it. Certain viruses or bacteria are more likely to infect cancer cells than normal cells, and such infected cancer cells may be targeted for the attack of immune cells. Accordingly, anticancer treatment methods have been proposed that intentionally use specific viral or bacterial infections to stimulate immune responses in the human body to fight against cancer cells. Infectious bacteria such as Shigella, Vibrio cholerae, and pathogenic E. coli invade intestinal cells, but do not reach the liver and spleen, which are important organs for triggering an immune response. In contrast, Salmonella may invade the spleen and liver through lymph nodes and stimulate a systemic immune response. Specifically, according to Leschner et al. (J. Mol. Med. 2010, 88, 763-773), CT26 tumor-bearing mice were intravenously injected with fluorescent Salmonella, and infection pathways were tracked over time. As a result, it was observed that, immediately after infection, the whole body of the mice was infected through the blood, Salmonella accumulated in the spleen and liver 20 minutes after infection, and Salmonella accumulated intensively only in tumor tissue 24 hours after infection.

However, Salmonella as a representative bacterium that causes food poisoning can cause life-threatening sepsis through infection, and thus it is too pathogenic to be used directly for cancer treatment. A research team at Yale University in the United States announced that, through genetic modification of Salmonella, Salmonella could be attenuated by removing its toxicity while maintaining its tumor-attacking properties, and that injection of the attenuated Salmonella may suppress tumors by inducing immune stimulation. However, the attenuated Salmonella is not only less viable and less able to induce immunity, but there is also a risk that mutations can cause such an attenuated strain to convert to wild-type Salmonella and cause sepsis. In addition, due to a lack of research and development funding, most anticancer treatments using attenuated Salmonella have been suspended or put on hold in Phase I in clinical trials.

The potential for causing sepsis associated with cancer treatment using bacteria such as Salmonella is a critical issue that must be overcome, and in addition, the stimulation of the immune response must also be actively achieved. Accordingly, research is mainly focused on developing strains that can additionally express or suppress substances beneficial to anticancer treatment, along with attenuation and immune activation, and using these strains in anticancer treatment. KR Patent Registration No: 10-0852687 discloses a Salmonella strain expressing tumor necrosis factor-alpha by transduction of a tumor necrosis factor-alpha protein vector into an attenuated Salmonella strain, and a composition for anticancer treatment, including the same, and KR 10-2021-0123556 discloses anticancer Salmonella that induces an immune response through filament growth regulation.

“Interleukin-21 (IL-21)” is widely involved in the differentiation and functions of lymphocytes and bone marrow cells, and simultaneously regulates innate immunity and adaptive immunity. In particular, in cancer treatment, IL-21 is known to have potential as an anticancer agent by enhancing the cancer cell-killing ability of CD8+ T cells and NK cells.

In this regard, the inventors of the present disclosure prepared a recombinant expression vector and an attenuated Salmonella strain transformed with the recombinant expression vector, the recombinant expression vector including: an flgM gene; an interleukin-21 gene; and an flhDC gene, and confirmed excellent cancer therapeutic effects of the recombinant expression vector, thereby completing the present disclosure.

The information set forth in this Background Art section is intended solely to enhance understanding of the background of the present disclosure, and may not include information that constitutes prior art, already known to a person skilled in the art to which the present disclosure pertains.

### DISCLOSURE OF INVENTION

### Technical Problem

An aspect provides a recombinant expression vector including: an flgM gene; an interleukin-21 gene; and an flhDC gene, wherein the flgM gene, the interleukin-21 gene, and the flhDC gene are operably linked to an inducible promoter.

Another aspect provides an attenuated Salmonella strain transformed with the recombinant expression vector.

Another aspect provides a pharmaceutical composition for cancer treatment, including the attenuated Salmonella strain as an active ingredient.

### Solution to Problem

An aspect provides a recombinant expression vector including: an flgM gene; an interleukin-21 gene; and an flhDC gene, wherein the flgM gene, the interleukin-21 gene, and the flhDC gene are operably linked to an inducible promoter.

The present disclosure relates to the recombinant expression vector in which the flgM gene and the interleukin-21 gene are directly linked or are linked to each other via a linker, such that the recombinant expression vector a gene encoding a fusion protein of flgM and interleukin-21. Hereinafter, in the present specification, a fusion protein of the flgM and the interleukin-21 is named “flgM-IL-21”.

In the present specification, the term “flgM” refers to a protein (or a gene encoding such a protein) that inhibits the o factor, which is responsible for recruiting a RNA polymerase required for late flagellar transcription in a type III secretion system. The “type III secretion system” refers to a pathogenic substance delivery system developed in Gram-negative bacteria. It is a multi-protein complex in the form of a syringe-shaped device that injects pathogenic active proteins directly into the cytoplasm through the membrane of a host cell (Mota L J et al, Ann Med. (2005); 37(4):234-249). In the type Ill secretion system, the flgM protein remains in the cytoplasm during the formation of the hook-basal body which is a basic structure capable of forming flagella on the cell membrane, and acts as an anti-σ²⁸ factor that inhibits the transcription of class III genes, such as flagellin subunit FliC or stator protein MotAB. Subsequently, after completion of the hook-basal body structure, a change in substrate specificity occurs simultaneously within a flagellar secretion system, causing flgM to be secreted out of the cell through the central pathway of the hook. Then, inside the cell, σ²⁸-dependent transcription begins. A secretion mechanism of flgM through the flagella formation process in the type III secretion system is shown in FIG. 2.

In the present specification term, “interleukin-21 (IL-21)” is a cytokine that has strong regulatory effects on the immune system, including natural killer (NK) cells and cytotoxic T cells that can destroy virus-infected cells or cancer cells. IL-21 is undergoing clinical trials as a single agent for the treatment of melanoma, renal cell carcinoma, and metastatic colorectal cancer, and is being studied as an anticancer agent in combination with agents such as cetuximab which is an antibody targeting a epidermal growth factor receptor (EGFR). A sequence of the IL-21 may be obtained from the NCBI GenBank, a publicly known database.

According to an embodiment, the present disclosure is to manufacture the recombinant expression vector including the flgM gene and the IL-21 gene, which is a protein to be secreted, so that a strain may secrete IL-21 outside the strain together with flgM through a type Ill secretion system.

In the present specification, the term “flhDC” refers to a major regulator of a flagellin operon gene, and flhDC can regulate the expression of the flgM gene. In an embodiment, the inclusion of the flhDC gene in the recombinant expression vector may contribute to enhancing the expression level or secretion level of the flgM-IL-21.

In the present specification, the term “gene” should be considered in its broadest sense, and a gene may be able to encode a structural protein or a regulatory protein. Here, the regulatory protein may include a transcription factor, a heat shock protein, or a protein involved in DNA/RNA replication, transcription, and/or translation.

In the present specification, the term “genetic structure” refers to an aggregate, functional unit, or a construct including the genetic information of a protein of interest, and may be considered in its broadest sense. The genetic structure may be, for the purpose of the present disclosure, intended to enhance the secretion of the flgM-IL-21 fusion protein from the attenuated Salmonella strain, and may refer to, for example, a DNA insert to which the flgM gene, a linker gene, and the IL-21 gene are operably linked.

In the present specification, the term “vector” refers to a DNA product containing a nucleotide sequence encoding a target polynucleotide operably linked to suitable regulatory sequences such that the target polynucleotide can be expressed in a suitable host. The regulatory sequences may include a promoter capable of initiating transcription, an optional operator sequence for regulating such transcription, a sequence encoding a suitable mRNA ribosome-binding site, and a sequence regulating termination of transcription and translation. Once transformed into a suitable host, the vector may be replicated or function regardless of the host genome, or may be integrated into the genome itself. The vector may be any one capable of replication in a host cell. For example, the vector may be a plasmid, cosmid, virus, or bacteriophage that is either native or recombinant. The vector may include a selection marker for selecting a host cell containing the vector. The selection marker is for selecting a cell transformed with the vector, and markers that confer a selectable phenotype, such as drug resistance, nutrient requirement, resistance to a cytotoxic agents, or expression of surface proteins, may be used. For example, the marker may be one or more selection markers selected from the group consisting of ampicillin, neomycin, puromycin, hygromycin, and zeocin. The vector may include genes involved in replication and/or copy number control, such as a replication origin, a promoter, or the like, and may include, although not limited thereto, a restriction enzyme site or the like.

In the present specification, the term “polynucleotide” may be in the form of RNA or DNA, and DNA may include cDNA and synthetic DNA. DNA may be single-stranded or double-stranded. In the case of a single strand, it may be a coding strand or a non-coding (antisense) strand, and a coding sequence may, as a result of degeneracy or redundancy of the genetic code, may encode the same polypeptide.

The polynucleotide may also include a variant of the polynucleotide described herein, wherein the variant of the polynucleotide may be a naturally occurring allelic variant of the polynucleotide or a non-naturally occurring variant of the polynucleotide. The allelic variant may be an alternate form of a polynucleotide sequence that may have substitution, deletion, or addition of one or more nucleotides, without substantially changing the function of a polynucleotide to be encoded. It is well known in the art that a single amino acid may be encoded by one or more nucleotide codons and that the polynucleotide may be easily modified to prepare an alternate polynucleotide encoding the same peptide.

In an embodiment, by including all the flgM gene, the IL-21 gene, and the flhDC gene, the recombinant expression vector may contribute to increasing the expression level or secretion level of the flgM-IL-21. For example, when the recombinant expression vector further includes the flhDC gene in addition to the flgM gene and the IL-21 in gene, the expression level or secretion level of the flgM-IL-21 may be increased.

In the present specification, the term “inducible promoter” refers to a promoter capable of switching the operation of a gene linked by an inducer, and examples thereof may include an ara promoter, a tac promoter, a lac promoter, a lacUV5 promoter, a lpp promoter, a pLλ promoter, a pRλ promoter, a rac5 promoter, an amp promoter, a recA promoter, a SP6 promoter, a trp promoter, a T7 promoter, a pBAD promoter, a Tet promoter, a trc promoter, a pepT promoter, a sulA promoter, a pol11 (dinA) promoter, a ruv promoter, a uvrA promoter, a uvrB promoter, a uvrD promoter, a umuDC promoter, a lexA promoter, a cea promoter, a caa promoter, a recN promoter, a pagC promoter, a hip promoter, an ansB promoter, or a pflE promoter.

In the present specification, the expression “operably linked” may refer that nucleotide sequences are linked on a single nucleic acid fragment such that one function is affected by the other.

In an embodiment, the expression of the flgM gene, the IL-21 gene, and the flhDC gene may be regulated by the same identical inducible promoter. In an embodiment, the promoter may be an ara promoter.

In an embodiment, due to the recombinant vector in which the expression of the flgM gene, the IL-21 gene, and the flhDC gene is regulated by the same inducible promoter, the expression ability or secretion ability of the flgM-IL-21 may be enhanced.

In an embodiment, the recombinant expression vector may include the flgM gene consisting of a polynucleotide sequence of SEQ ID NO: 1, the IL-21 gene consisting of a polynucleotide sequence of SEQ ID NO: 2, and the flhDC gene consisting of a polynucleotide sequence of SEQ ID NO: 3.

In an embodiment, the flgM gene and the IL-21 gene may be linked to each other via a linker.

In the present specification, the term “linker” refers to a polypeptide chain used to link a heterologous domain to a protein of interest, or a gene encoding the polypeptide chain. A linker peptide may have 2 to 50 amino acids in length. For example, although not limited thereto, the linker peptide may have, in length, 2 to 40 amino acids, 2 to 30 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 20 amino acids, 5 to 15 amino acids, or 5 to 10 amino acids. For example, the linker peptide may be, although not limited thereto, at least one selected from the group consisting of (GₗSₘ)ₙ (where I, m, and n are each ≥2), (GₗSₘ)ₙ—Hₚ-(GₗSₘ)ₙ (where l, mm and n are each ≥1, H≥6), (G₄S)ₐ(EAAAK)_{b}(G₄S)ₐ (where a and b are each an integer from 1 to 4), (G₄S)ₚ(EAAAK)_{q} (where p and q are each an integer from 1 to 4), (EAAAK)ₓ(G₄S)_{y} (where x and y are each an integer from 1 to 4), A(EAAAK)₄ALEA(EAAAK)₄A, (GSSGGS)ᵢ (where i is an integer from 1 to 4), KESGSVSSEQLAQFRSLD, EGKSSGSGSESKST, GSAGSAAGSGEF, (EAAAK) k (where k is an integer from 1 to 5), CRRRRRREAEAC, GGGGGGGG, GGGGGG, AEAAAKEAAAAKA, PAPAP, VSQTSKLTRAETVFPDV, PLGLWA, TRHRQPRGWE, AGNRVRRSVG, RRRRRRRR, GGSSHHHHHHSSGG, GGSSHHHHHHHHSSGG, and GSSGGSGSSGGSGGGDEADGSRGSQKAGVDE. In an embodiment, the linker peptide may be GGSSHHHHHHHHSSGG.

The recombinant expression vector according to an embodiment may have enhanced ability to express or secrete the flgM-IL-21 by linking the flgM gene and the IL-21 gene through a gene (SEQ ID NO: 4) encoding the GGSSHHHHHHHHSSGG linker peptide.

Another aspect provides an attenuated Salmonella strain transformed with the recombinant expression vector. Regarding the attenuated Salmonella strain, the same terms or elements as those already mentioned are as described above.

In the present specification, the term, “transformation” refers to introducing a vector including a polynucleotide encoding a target protein into a host cell to enable the expression of the protein encoded by the polynucleotide within the host cell. For example, the polynucleotide may be introduced in the form of an expression cassette into a host cell, or may be introduced in its own form into a host cell, so as to be operably linked to a sequence necessary for the expression in the host cell. However, the polynucleotide is not limited thereto.

In the present specification, the term “attenuated” refers to artificially weakening the virulence of a living pathogen. In detail, it means that, by mutating genes involved in the essential metabolism of the pathogen, only the immune system is stimulated without causing a disease in the body, thereby inducing immunity. Genes causing attenuation of Salmonella are well known in the art, and the attenuated Salmonella strain may be, for example, one in which the function of one or more genes selected from the group consisting of aroA, aroC, aroD, aroE, asd, Rpur, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rcsB, rfc, poxA, galU, cdt, pur, ssa, guaA, guaB, fliD, flgK, flgL, relA, and spoT has been lost. To prevent reversion to the wild type upon in vivo application and to further attenuate virulence, the function of two or more of the genes above may be lost. The attenuated Salmonella strain may have anticancer activity by targeting a tumor site and inducing an immune response.

The attenuated Salmonella strain according to an embodiment may be transformed with the recombinant expression vector, and it was confirmed that the expression level and secretion level of the flgM-IL-21 were significantly enhanced, thereby confirming that an agent including the Salmonella strain exhibits an excellent anticancer effect when administered to an individual.

In an embodiment, the attenuated Salmonella strain may be one in which an asd gene, a rcsB gene, and a galE gene have been lost. In detail, the attenuated Salmonella strain may be one in which the function of an aroA gene and a aroD gene has been additionally lost so that an aroA gene, an aroD gene, an asd gene, a rcsB gene, and a galE gene are lost.

The attenuated Salmonella strain according to an embodiment may be one in which the expression of an attenuated gene is regulated by a second inducible promoter. That is, a specific gene may be regulated for loss of function by a second inducible promoter. In detail, the attenuated Salmonella strain may further include a second inducible promoter upstream of the attenuated gene to enable switching the attenuation. For example, when the attenuated Salmonella strain is injected in an attenuated state into a subject and targeting a tumor site is completed, the attenuated Salmonella strain may be reverted to wild-type Salmonella by operation of the second inducible promoter so that high immune activity compared to the attenuated Salmonella with low immune activity is resulted, enabling cancer treatment more effectively.

In an embodiment, the attenuated Salmonella strain may have lost a galE gene, and loss-of-function of the galE gene may be covered by switch-of-function of a tetRA promoter. Therefore, after the attenuated Salmonella strain is injected in an attenuated into a subject and targeted a tumor site, the expression of the galE gene is induced by doxycycline, leading to induction of high immune activity of the Salmonella strain. In the present specification, the loss-of-function regulation of the galE gene is expressed as galE:tetRA.

In the case of the attenuated Salmonella strain according to an embodiment, the asd gene, the rcsB gene, and the galE gene may have loss-of-function, and it is accordingly confirmed that the attenuated Salmonella strain has excellent ability to express or secrete the flgM-IL-21.

In an embodiment, the attenuated Salmonella strain may have enhanced secretion ability for the IL-21 protein. The attenuated Salmonella strain according to an embodiment does not merely express a peptide component having anticancer properties or toxicity properties against cancer cells, but is capable of secretion a peptide component expressed in a targeted site, thereby exhibiting efficacy as a targeted anticancer therapeutic agent.

According to an embodiment, when the attenuated Salmonella strain is transformed with the recombinant expression vector, the expression of the IL-21 may be regulated in the presence of arabinose, and the flgM of the type III secretion system may be used to effectively secrete the IL-21 protein outside the strain. In addition, the expression of flhDC may be also regulated by the same inducible promoter so that the expression level of the IL-21 secreted extracellularly may be increased.

In an embodiment, the attenuated Salmonella strain may be one deposited with Accession No. KCTC15503BP.

Another aspect provides a pharmaceutical composition for anticancer treatment or a pharmaceutical composition for preventing or treating cancer, each including the attenuated Salmonella strain as an active ingredient.

Regarding the pharmaceutical composition, the same terms or elements as those already mentioned are as described above.

“Cancer” which is a target disease to be prevented or treated by the composition of the present disclosure is meant to be a collective term for diseases caused by cells that have aggressive characteristics of dividing and growing beyond normal growth limits, invasive characteristics of infiltrating surrounding tissues, and metastatic characteristics of spreading to other parts of the body. The cancer may be, for example, a solid cancer. Preferably, the cancer may be expanded without limitation as long as it is a tumor including cancer tissue or cancer cells in which the cancer-targeting ability of the Salmonella strain can be exerted. For example, the type of cancer may be, although not limited thereto, any one selected from the group consisting of pancreatic cancer, colorectal cancer, colon cancer, stomach cancer, liver cancer, brain cancer, breast cancer, thyroid cancer, bladder cancer, esophageal cancer, head and neck cancer, skin cancer, uterine cancer, and lung cancer.

The composition of the present disclosure may be prepared by a method known in the pharmaceutical field for use as a drug fur preventing or treating cancer, and may be used on its own or in a mixture with a pharmaceutically acceptable carrier, an excipient (e.g., a forming agent), a diluent, and the like.

In the present specification, a “pharmaceutically acceptable carrier” may be determined in part by a particular composition being administered, as well as in part by a particular method used to administer the composition. Therefore, suitable formulations of the composition may be very diverse, and the composition may be, for example, formulated for parenteral (i.e., intramuscular, intradermal, or subcutaneous) administration or nasopharyngeal (i.e., intranasal) administration. In general, formulations for parenteral administration may include sterilized aqueous solution or non-aqueous solution, suspensions, and emulsions. Examples of the non-aqueous solvent may include propylene glycol, polyethylene glycol, plant oil such as olive oil, and injectable organic esters such as ethyl oleate. An aqueous carrier may include water, alcohol/aqueous solutions, emulsions, or suspensions, in addition to saline and buffered media. Parenteral vehicles may include sodium chloride solution, Ringer's dextrose, dextrose and sodium chloride, lactated Ringer's solution, fixed oils. Preservatives and other additives may be also present in the form of, for example, antibacterial agents, antioxidants, chelating agents, inert gases, and the like.

A dosage of the active ingredient according to the present disclosure may be appropriately selected depending on the degree of absorption of the active ingredient in the body, the type of formulations, the age, gender, and condition of a patient, the degree of symptoms, and the like, and the active ingredient, and the active ingredient may be administered once a day or divided into several times. A typical dosage is 0.001 mg/kg·day to 10 g/kg·day.

Another aspect provides a method of preventing or treating cancer, the method including administering to a subject an anticancer pharmaceutical composition including the attenuated Salmonella strain as an active ingredient in an amount effective for preventing or treating cancer.

Regarding the method of preventing or treating cancer, the same terms or elements as those already mentioned are as described above.

The subject may be a mammal. The mammal may be a human, a mouse, a rat, a horse, a dog, a cat, a cow, a goat, or a pig.

The administration may be done by any common route as long as the composition can reach a target tissue. For example, the administration may be done by routes such as eye drop administration, intraperitoneal administration, intravenous administration, intramuscular administration, subcutaneous administration, intradermal administration, transdermal patch administration, oral administration, intranasal administration, intrapulmonary administration, and rectal administration, and specifically, the administration may be done as intended by a route of eye drop administration.

In the present specification, the term “treatment” or “treating” or “alleviating” or “ameliorating” is used interchangeably. These terms refer to methods of obtaining beneficial or desired results including, but not limited to, therapeutic benefits and/or prophylactic benefits. Therapeutic benefit means any therapeutically relevant improvement in or effect on one or more diseases, illnesses, or symptoms under treatment. For prophylactic benefits, the composition may be administered to a subject at risk of developing a particular disease, illness, or symptom, or to a subject reporting one or more physiological symptoms of a disease, even though a disease, illness or symptom has not yet manifested.

As used in the present specification, the term “effective amount” or “therapeutically effective amount” refers to an amount of an agent that is sufficient to cause a beneficial or desired result. The therapeutically effective amount may vary depending on one or more of a subject and pathological conditions being treated, the weight and age of a subject, severity of a pathological condition, administration methods, etc., which may be readily determined by a person skilled in the art. In addition, the term is applied to a dose that will provide an image for detection by any one of imaging methods described herein. A specific dose may vary depending on one or more of a particular agent chosen, a dosing regimen to be followed, whether it is administered in combination with other compounds, timing of administration, a tissue to be imaged, and a physical delivery system carrying the tissue.

### Advantageous Effects of Invention

According to the recombinant expression vector of an aspect, it was confirmed that the expression and secretion of the flgM-IL-21 were significantly enhanced in the attenuated Salmonella strain transformed with the recombinant expression vector.

Also, according to the recombinant expression vector of an aspect, it was confirmed that excellent anticancer effects and excellent stability were exhibited upon administration of a formulation including the attenuated Salmonella strain transformed with the recombinant expression vector.

Therefore, the recombinant expression vector of an aspect and the attenuated Salmonella strain transformed therewith may be used as active ingredients in an anticancer composition.

### BRIEF DESCRIPTION OF DRAWINGS

FIG. 1 is a schematic diagram showing a process of extracellular secretion of flgM-IL-21, using a type III secretion system.

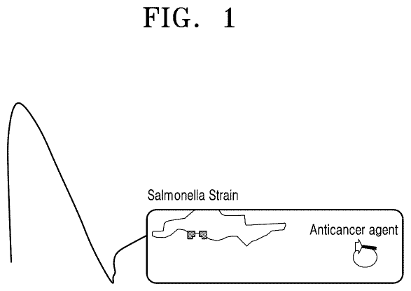

FIG. 2 is a schematic diagram showing a flgM secretion mechanism using a flagella formation process in a type III secretion system.

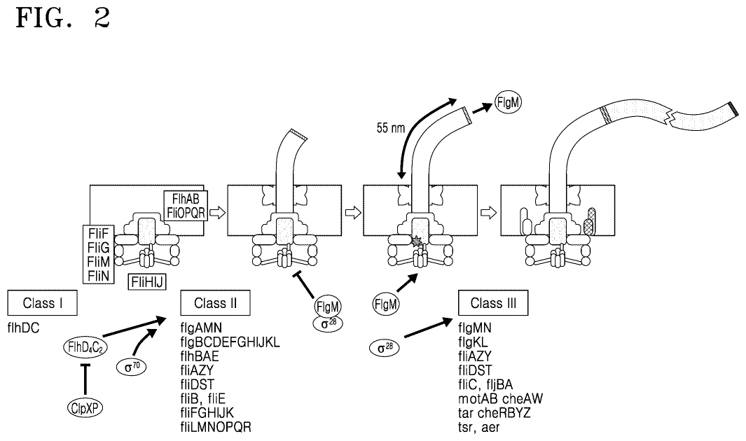

FIG. 3 is a diagram showing a cleavage map of a flgM-linker-IL-21-flhDC-pBAD18-asd+ plasmid according to an embodiment.

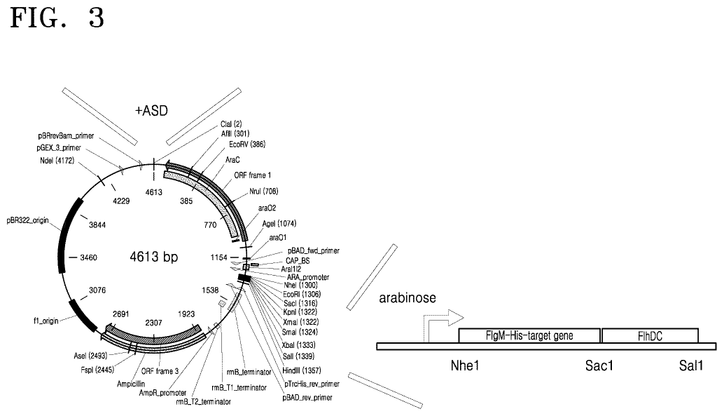

FIG. 4 shows the results of confirming the protein secretion level of an attenuated Salmonella strain transformed with a flgM-linker1-IL-21-flhDC-pBAD18/flgM-linker2-IL-21-flhDC-pBAD18 plasmid according to an embodiment. The secretion level of a flgM-IL-21 protein was confirmed in a whole cell culture solution as shown in lanes 1 and 2, in pellets as shown in lanes 3 and 4, and in supernatant as shown in lanes 5 and 6.

FIG. 5 is a schematic diagram illustrating a process of treating a tumor mouse model with a transformed attenuated Salmonella strain according to an embodiment.

FIG. 6 is a graph showing changes in tumor volume in a mouse when treated with a transformed attenuated Salmonella strain according to an embodiment (PBS: control, uninduction: non-arabinose treated group, induction: arabinose treated group).

FIG. 7 is a graph showing changes in body weight of a mouse when treated with a transformed attenuated Salmonella strain according to an embodiment (PBS: control, uninduction: non-arabinose treated group, induction: arabinose treated group).

### BEST MODE FOR CARRYING OUT THE INVENTION

### Mode for the Invention

Hereinafter, the present disclosure will be described in more detail with reference to Examples below. However, these Examples are for illustrative purposes only, and the scope of the present disclosure is not intended to be limited by these Examples.

### Example 1: Preparation of Plasmid for Secretion of IL-21

### 1-1. Preparation of Insert DNA

To prepare a plasmid for secretion of IL-21, a genetic structure in which an flgM gene and an IL-21 gene are linked was prepared.

Specifically, the flgM DNA sequence of wild-type Salmonella (flgM anti-sigma-28 factor FlgM [Salmonella enterica subsp. enterica serovar Typhimurium str. LT2] Gene ID: 1252690, Locus tagSTM1172 NC_003197.2 (1257036 . . . 1257341, complement)) and a DNA sequence obtained through codon optimization (Salmonella) for IL-21 were linked to prepare a genetic structure according to an embodiment. For codon optimization of the IL-21, 130 amino acids were substituted with DNA and TAA was inserted at the end.

The flgM DNA sequence and the IL-21 DNA sequence were linked via a linker, and to enhance the expression of the flgM gene, a 15-base Shine-dalgarno sequence (AGGAGGTTTGATCCT) was inserted upstream of the flgM gene. Also, considering cloning, an Nhe I site was inserted at the front and a Sac I site was inserted following the stop codon of the IL-21. Meanwhile, the genetic information of major structures in this Example is as shown in Table 1.

TABLE 1

| | | SEQ |
|---|---|---|
| Name | Sequence information | ID NO: |
| flgM | ATGAGCATTGACCGTACCTCACCTTTGAA | 1 |
| | ACCCGTTAGCACTGTCCAGACGCGCGAAA | |
| | CCAGCGACACGCCGGTACAAAAAACGCGT | |
| | CAGGAAAAAACGTCCGCCGCGACGAGCGC | |
| | CAGCGTAACGTTAAGCGACGCGCAAGCGA | |
| | AGCTCATGCAGCCAGGCGTCAGCGACATT | |
| | AATATGGAACGCGTCGAAGCATTAAAAAC | |
| | GGCTATCCGTAACGGTGAGTTAAAAATGG | |
| | ATACGGGAAAAATAGCAGACTCGCTCATT | |
| | CGCGAGGCGCAGAGCTACTTACAGAGTAA | |
| | AAAA | |
| IL-21 | ATGCACAAATCTTCTCCGCAGGGTCCGGA | 2 |
| | TCGTCTGCTGATCCGTCTGCGTCACCTGA | |
| | TCGATATCGTTGAACAGCTGAAAATCTAC | |
| | GAAAACGACCTGGATCCGGAACTGCTGTC | |
| | TGCTCCGCAGGATGTTAAAGGTCACTGCG | |
| | AACACGCTGCTTTCGCGTGTTTCCAGAAA | |
| | GCTAAACTGAAACCGTCTAATCCGGGTAA | |
| | CAACAAAACCTTCATCATCGACCTGGTTG | |
| | CGCAGCTGCGTCGTCGTCTGCCGGCTCGT | |
| | CGTGGTGGTAAAAAACAGAAACACATCGC | |
| | TAAATGCCCGTCTTGTGACTCTTACGAAA | |
| | AACGTACCCCGAAAGAATTCCTGGAACGT | |
| | CTGAAATGGCTGCTGCAGAAAATGATCCA | |
| | CCAGCACCTGTCTTAA | |
| linker 1 | GGCGGCAGCAGCCATCACCATCACCATCA | 4 |
| | CCATCACAGCAGCGGCGGC | |
| linker 2 | GGCGGCAGCAGCCATCACCATCACCATCA | 5 |
| | CAGCAGCGGCGGC | |

By using the structure, genetic structures of flgM-linker1-IL-21 (SEQ ID NO: 6) and flgM-linker2-IL-21 (SEQ ID NO: 7) were obtained through the gene synthesis service of Bionics Co., Ltd.

Then, the genetic structure was reacted with 10 U of each of enzymes, Nhe I and Sac I, at 37° C. for 2 hours. After performing electrophoresis on the reaction product, DNA of about 500 bp was identified by using a gel extraction kit (by Qiagen Co., Ltd.), and a flgM-linker1-IL-21 insert and a flgM-linker2-IL-21 insert were obtained.

Afterwards, to obtain flhDC insert DNA, PRC was performed by using the genomic DNA of wild-type Salmonella LT2 as a template and flhD-sac1-F and flhC-Sal1-r shown in Table 2 as primers, and a PCR product including the flhDC gene was obtained. The specific PCR conditions are as follows: 5 μl of 10× buffer, 10 μl of 5×Q solution, 7.5 μl of 2 mM dNTPs, 2.5 μl of 20 μm flhD-sac1-f primer, 2.5 μl of 20 μm flhC-Sal1-r primer, 1 μl of Taq polymerase, 2 μl of 10 ng/μl genomic DNA, and 19.5 μl of water were mixed; and PCR was performed thereon by using Qiagen system at 95° C. for 5 minutes, followed by 30 cycles of PCR at 94° C. for 30 seconds, at 49° C. for 30 seconds, and at 72° C. for 1 minute, and then at 72° C. for 10 minutes. 1 μg of the PCR fragment purified by a PCR purification kit was reacted with 10 U of each of sac I and Sal I at 37° C. for 2 hours, and the reaction product was then purified by a PCR purification kit to prepare flhDC insert DNA (SEQ ID NO: 3).

TABLE 2

| | | SEQ |
|---|---|---|
| Name | Sequence information | ID NO: |
| flhD-Sac1-f | gatcgatcgagctcaggaggtttgat | 8 |
| | cctatgggaacaatgcatacatccga | |
| | gttgct | |
| flhC-Sal1-r | gatcgatcgtcgacttaaacagcctg | 9 |
| | ttcgatctgttcatccagcagtt | |

### 1-2. Preparation of Plasmid

Next, a pBAD18 asd+ plasmid was used as a vector and reacted with 10 U of each enzyme, Nhe I and Sac I, at 37° C. for 2 hours. Then, the reaction product was purified by a PCR purification kit to obtain each vector DNA. The asd gene inserted into the plasmid may consist of the nucleotide sequence of SEQ ID NO: 10. Then, each vector DNA was ligated with the flgM-linker-IL-21 insert DNA at 25° C. for 30 minutes, and transformed into DH5a competent cells. Next, the transformed cells were spread onto a LB ampicillin (amp) solid medium and cultured at 37° C., and colonies having antibiotic resistance were selected. Six candidate groups were selected from the selected colonies and reacted with 10 U of each of Nhe I and Sac I at 37° C. for 1 hour. Then, generation of bands of about 800 bp was confirmed through electrophoresis. In addition, the nucleotide sequence analysis was performed on the candidate groups for the plasmid to confirm the insertion of the flgM, the linker, and the IL-21 gene.

Then, 1 μg of each plasmid was reacted with 10 U of sac I and Sal I at 37° C. for 2 hours, and the reaction product was purified by a PCR purification kit, thereby completing the preparation of vector DNA. Then, each vector DNA was ligated with the flhDC insert DNA at 25° C. for 30 minutes and transformed into DH5a competent cells. The transformed cells were spread onto a LB amp solid medium and cultured at 37° C., and colonies having antibiotic resistance were selected. Six candidate groups were selected from the selected colonies and reacted with 10 U of each of sac I and Sal I at 37° C. for 1 hour. Then, generation of bands was confirmed through electrophoresis. The nucleotide sequence analysis was performed on the candidate groups to confirm insertion of the flhDC gene.

Accordingly, the flgM-linker1-IL-21-flhDC-pBAD18-asd+/flgM-linker2-IL-21-flhDC-pBAD18-asd+ plasmid were prepared.

### Example 2: Preparation of Attenuated Salmonella Strain Secreting flgM-IL-21

An attenuated Salmonella strain was transformed with the plasmid prepared in Example 1. For candidate groups for the attenuated Salmonella strain, BRD509 asd galE:tetRA-(#1317) and BRD509 asd rcsB galE:tetRA-(#1323) strains were used, wherein these strains were provided by Chungnam National University. The BRD509 Salmonella strain had loss-of-function of an aroA gene and an aroD gene. The genetic information and strain information of the transformed strains are as shown in Table 3.

TABLE 3

| | | Comparison | Comparison | Comparison |
|---|---|---|---|---|
| | Test group | group 1 | group 2 | group 3 |
| Linker | Linker1 | Linker2 | Linker1 | Linker2 |
| Strain | #1323 | #1323 | #1317 | #1317 |

### Example 3: Confirmation of Protein Expression and Secretion Ability

To confirm the ability of the attenuated Salmonella strain transformed with the recombinant expression vector prepared according to an embodiment for the expression and secretion of the flgM-IL-21, the expression level of the protein was measured through western blotting by the following methods.

A single colony of each of the transformed strains was inoculated into a LM amp liquid medium and cultured with shaking at 37° C. for 12 to 16 hours. Afterwards, the culture was diluted in a fresh LB amp liquid medium until OD600 reached 0.05, and then cultured with shaking at 37° C. for 2 hours. When the OD600 reached 0.4 to 0.6 by shaking culture, arabinose was treated at a concentration of 0.2% (w/v), and the resulting culture was additionally cultured by shaking at 37° C. for 5 hours.

Afterwards, 1 ml of the culture solution of the whole cell (lanes 1 and 2) and another 1 ml of the culture solution of the whole cell were centrifuged, and the pellets (lanes 3 and 4) were separated from the supernatant (lanes 5 and 6) to prepare samples. The strains were divided into those not treated with the arabinose at a concentration of 0.2% (w/v) (final) (lanes 1, 3, and 5) and those treated with the arabinose (lanes 2, 4, and 6), and then cultured. The culture solution was centrifuged at 8000 rpm to prepare each sample. The supernatant was filtered through a 0.2 μm filter to completely remove the bacteria, and the filtrate was concentrated with Nanosep (OD010C35 3K omega). 50 μl of each of the obtained samples was mixed with a SDS sample buffer, denatured for 5 minutes in a heating block at 100° C., and centrifuged again at 13000 rpm at 4° C. for 5 minutes. Then, electrophoresis was performed thereon on a 10% polyacrylamide gel. The gel was transferred by using polyvinylidene fluoride (PVDF) to transfer the protein onto a PVDF membrane. The membrane was then blocked with a blocking buffer at room temperature for 1 hour. Afterwards, the primary antibody anti-his tag (SB194b, Southern Biotech) was used, diluted at 1/1000, and reacted at 4° C. for 16 hours. Afterwards, the membrane was washed three times with tris-buffered saline with 0.1% Tween-20 (TBST) at 10 minute-intervals, and then allowed for a reaction with the anti-mouse-IgG (#7076s, Cell signaling, Danvers, MA, USA) secondary antibody at room temperature for 1 hour. Following the reaction, the membrane was washed with TBST at 10 minute-intervals, sensitized to an X-ray film by using an ECL buffer, and then developed to confirm the expression level of each protein.

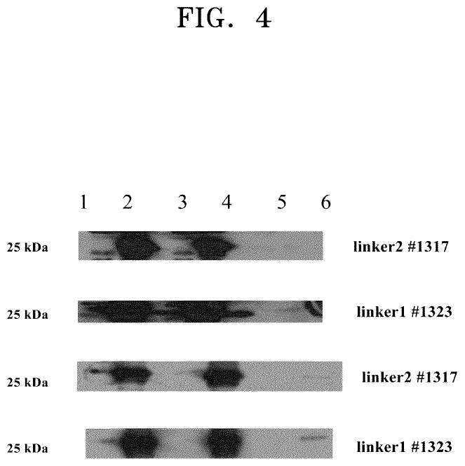

In FIG. 4, lanes 2, 4, and 6 show the samples treated with the arabinose (final concentration of 0.2% (w/v)), and lanes 1, 3, and 5 show the samples not treated with the arabinose. Also, lanes 1 and 2 show the results from the culture solution of the whole cell, lanes 3 and 4 show the results from the pellets, and lanes 5 and 6 show the results from the supernatant.

As shown in FIG. 4, the samples of lanes 2, 4, and 6 treated with the arabinose expressed a larger amount of the flgM- IL-21 at the same concentration than the samples of lanes 1, 3, and 5 not treated with the arabinose. Also, the highest amount of the flgM-IL-21 was confirmed in lane 6 (supernatant) in #1323 strain transformed with the recombinant expression vector to which the linker 1 was inserted.

Based on the results, it was confirmed that the expression of the flgM-IL-21 was regulated by the arabinose, and that the flgM-linker1-IL-21-flhDC-pBAD18-asd+/BRD509 asd rcsB galE:tetRA-(#1323) strain had the best ability to secrete extracellular proteins without cell rupture.

Accordingly, the inventors of the present disclosure deposited the flgM-linker1-IL-21-flhDC-pBAD18-asd+/BRD509 asd rcsB galE:tetRA-(#1323) strain of the test group to the Korean Collection for Type Cultures of Korea Research Institute of Bioscience and Biotechnology on Jul. 14, 2023, and the strain was assigned the Accession No: KCTC15503BP (S-flgM-mIL21-flhDC-pBAD18-ASD+/#1323).

### Example 4: Evaluation of Anticancer Effect Using Mouse Model

4-1. Preparation of Mouse Model with Xenografted Tumor Cells

In this example, 6-week-old BALB/c female mice weighing about 20 g were purchased and used as experimental animals. All animal care, experiments, and euthanasia were performed according to the approved protocols. Murine CT26 colon cancer cells were purchased from the American Type Culture Collection (ATCC) and cultured in a DMEM medium supplemented with 10% FBS and 1% penicillin-streptomycin. The CT26 tumor cells cultured in vitro were harvested and suspended in 20 μl of PBS, and 5×10⁵ cells were injected subcutaneously into the right back of the BALB/c mouse to perform xenografting of the tumor cells. After about 2 weeks, when the tumor size reached 300 mm³, the flgM-linker1-IL-21-flhDC-pBAD18-asd+/BRD509 asd rcsB galE:tetRA-(#1323) strain was diluted in PBS and injected through the tail vein at a dose of about 1×10⁷ CFU/mice (Day 0).

### 4-2. Confirmation of Anticancer Effects

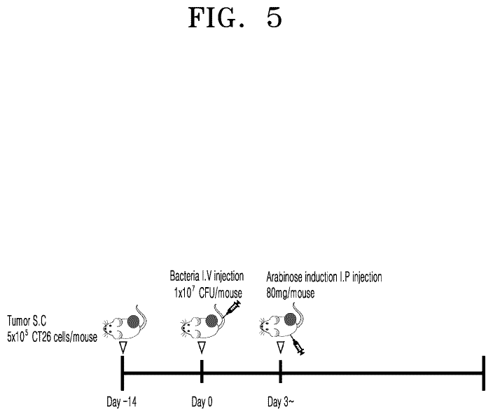

Then, to induce the expression or secretion of the flgM-IL-21, 80 mg/mice of L-arabinose was injected intraperitoneally into the mouse every day, and the body weight of the mouse and the tumor size were measured and recorded once every two days. Here, the tumor volume was calculated by the following equation (tumor length×tumor width×tumor height)/2, and the mouse was euthanized when the tumor size exceeded 1,500 mm³⁰ according to the guidelines of the Animal Experiment Ethics Committee. FIG. 5 is a schematic diagram illustrating a treatment process for a tumor mouse model of an attenuated Salmonella strain transformed according to an embodiment.

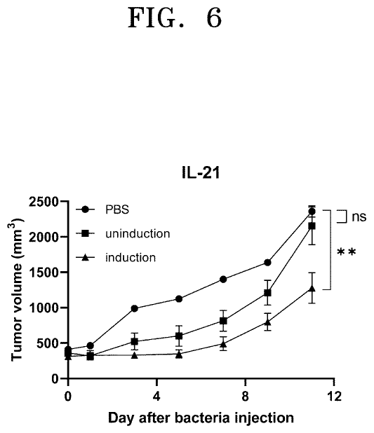

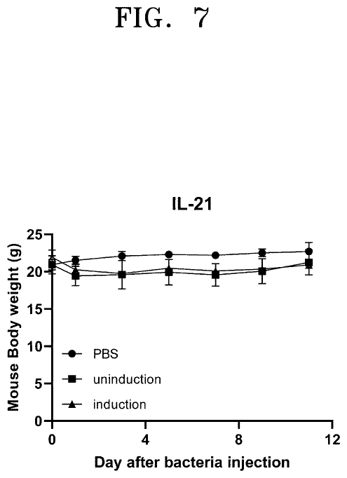

As shown in FIGS. 6 and 7, it was confirmed that the attenuated Salmonella strain transformed according to an embodiment significantly inhibited the tumor growth in the group (induction) in which the expression or secretion of the IL-21 was induced by arabinose. Also, it was confirmed that the side effects were minimal as the weight change of the mouse was not significant while showing excellent anticancer effects.

Accordingly, it was confirmed that the attenuated Salmonella strain transformed according to an embodiment is safe and has excellent anticancer effects, and thus can be utilized as an active ingredient for the treatment of cancer.

While the foregoing has described certain aspects of the present disclosure, it will be apparent to those skilled in the art that these specific techniques are merely preferred embodiments and are not intended to limit the scope of the present disclosure. Therefore, the substantial scope of the present disclosure will be defined by the appended claims and equivalents thereof.

### [Accession No]

Name of depository institution: Korean Collection for Type Cultures (KCTC) of
Korea Research Institute of Bioscience and Biotechnology
Accession No: KCTC15503BP
Accession Date: Jul. 14, 2023

1. A recombinant expression vector comprising: an flgM gene; an interleukin-21 gene; and an flhDC gene, wherein the flgM gene, the interleukin-21 gene, and the flhDC gene are operably linked to an inducible promoter.

2. The recombinant expression vector of claim 1, wherein expression of the flgM gene, the interleukin-21 gene, and the flhDC gene are is regulated by the same inducible promoter.

3. The recombinant expression vector of claim 1, wherein the recombinant expression vector comprises the flgM gene consisting of a polynucleotide sequence of SEQ ID NO: 1, the interleukin-21 gene consisting of a polynucleotide sequence of SEQ ID NO: 2, and the flhDC gene consisting of a polynucleotide sequence of SEQ ID NO: 3.

4. The recombinant expression vector of claim 1, wherein the flgM gene and the interleukin-21 gene are linked via a linker.

5. The recombinant expression vector of claim 4, wherein the linker consists of a polynucleotide sequence of SEQ ID NO: 4.

6. An attenuated Salmonella strain transformed with the recombinant expression vector of claim 1.

7. The attenuated Salmonella strain of claim 6, wherein the attenuated Salmonella strain has loss-of-function in an asd gene, an rcsB gene, and a galE gene.

8. The attenuated Salmonella strain of claim 6, wherein the attenuated Salmonella strain has enhanced ability to secrete an interleukin-21 protein.

9. The attenuated Salmonella strain of claim 6, wherein the attenuated Salmonella strain is deposited with Accession No. KCTC15503BP.

10. A pharmaceutical composition for cancer treatment, comprising the attenuated Salmonella strain of claim 6 as an active ingredient.
